(19) United States
(12) Patent Application Publication
(10) Pub. No.: US 2012/0150085 A1
(43) Pub. Date: Jun. 14, 2012

(54) SPINE-ALIGN GARMENT SYSTEMS

(76) Inventor: Andrew Kayser, West Newton, MA (US)

(21) Appl. No.: 13/323,011

(22) Filed: Dec. 12, 2011

Related U.S. Application Data
(60) Provisional application No. 61/422,558, filed on Dec. 13, 2010.

(51) Int. Cl. A61F 5/00; A61F 5/02

(52) U.S. Cl. 602/19; 2/44

(57) ABSTRACT

A posture-supporting, spine-aligning garment to support and correct body alignment and general positioning in its wearer-users; the garment being aesthetically pleasing. Posture-support, spine-align shirt with “Backboard” Technology is designed to alleviate back pain by promoting proper lumbar and thoracic alignment, core stabilization, corrective shoulder positioning, and upper body support. Posture-support spine-align garment system comprises an upper body apparel assembly having an elastic retention system; flexible stretch bands; anchors; a backboard, spine-specific support; and at least one fabric cover. The device may be manufactured with all or without one or more of the mentioned items. Posture-supporting, spine-aligning garment may comprise a posture shirt in preferred embodiments.

### CROSS-REFERENCE TO RELATED APPLICATION

The present application is related to and claims priority from prior provisional application Ser. No. 61/422,558, filed Dec. 13, 2010 which application is incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

The following includes information that may be useful in understanding the present invention(s). It is not an admission that any of the information provided herein is prior art, or material, to the presently described or claimed inventions, or that any publication or document that is specifically or implicitly referenced is prior art.

### BACKGROUND OF THE INVENTION

1. Field of the Invention

The present invention relates generally to the field of garments and more specifically relates to posture-supporting spine-aligning garments.

2. Description of the Related Art

Many individuals in modern society suffer from chronic low back, mid back and shoulder pain issues. Some individuals rely on medication to help relieve their symptoms, while others feel there is nothing they can do on their own to help alleviate the pain so they rely on chiropractors, physical therapists, acupuncturists, cranial-sacral therapists, and other specialists (cortisone shots and facet-joint blocks). Relying on medication, even if physician prescribed, may lead to addiction and typically only serves to prevent the symptoms. Although back and shoulder pain may be a result of numerous events and conditions, such as occupational stress, poor posture requiring additional support, post-surgical pain, muscular strains, spondylolisthesis, osteoporosis, and lumbar disc herniation (protrusion) or degeneration, the conventional chiropractic and other therapeutic methods, systems and devices may be unsuitable to correct the above-mentioned problems. Further, treatments from chiropractors and other specialists may prove expensive and an inconvenience.

Spine braces are used for a variety of reasons including: controlling pain, lessening the chance of further injury, allowing healing to take place, compensating for muscle weakness, or preventing deformities. Braces may be used such as the Milwaukee brace and the Boston brace by those with scoliosis. The Milwaukee brace for example has a pelvic girdle, two posterior uprights, one anterior upright, and a ring around the base of the skull that also supports the lower jaw. Originally made of leather and metal, it has been revised now and is constructed out of rigid plastic with metal uprights. Pads with straps attached to the frame are used to apply corrective forces. Studies show that although bracing may control the spinal curve and prevent progression; however in most cases, bracing does not correct the curve; it just keeps it from getting worse and is uncomfortable to wear. Additionally, these braces may be unsightly which may cause embarrassment in the user-wearers.

Various attempts have been made to solve the above-mentioned problems such as those found in U.S. Pat. Nos. 7,568,966; 1,367,420; 4,870,706; 5,728,055; 3,282,264; 7,134,969; D323,909; 3,292,616; 5,537,690; and 2003/0130603. This art is representative of back treatment and/or support means. None of the above inventions and patents, taken either singly or in combination, is seen to describe the invention as claimed.

Ideally, a posture-support spine-align garment system should be comfortable, aesthetically pleasing, operate reliably and be manufactured at a modest expense. Thus, a need exists for a reliable posture-support spine-align garment system to avoid the above-mentioned problems.

### BRIEF SUMMARY OF THE INVENTION

In view of the foregoing disadvantages inherent in the known garment art, the present invention provides a novel posture-supporting, spine-aligning garment system. The general purpose of the present invention, which will be described subsequently in greater detail is to provide an aesthetically pleasing posture-supporting, spine-aligning garment to support and correct body alignment and general positioning in its wearer-users. Posture-Support Spine-Align Posture Shirt with “Backboard” Technology (posture-support, spine-align garment system) is designed to alleviate back pain by promoting proper lumbar and thoracic alignment, core stabilization, corrective shoulder positioning, and upper body support. The posture-support spine-align garment system may also be used as a preventive measure. The posture-support spine-align garment systems are comfortable, easy to wear/use, and provide custom support of the entire upper body. Both sections of the trunk and torso require support to in turn accommodate and support each other.

A posture-support spine-align garment system is disclosed herein, a preferred embodiment comprising: a lumbar (and thoracic) support garment for surrounding at least the lumbar (and lower thoracic) region of a user-wearer of the lumbar support garment. The lumbar support garment preferably comprises a main cloth (including back and front fabric covers); anchors; elastic support system (may be used in any areas, such as the lower sternum, obliques, abdomen, clavical, etc. to provide support); and a backboard or spine support. The main cloth may comprise a single-sheet of cloth made of stretchable fabric and a lining cloth made of stretchable knit fabric, which extends in use from a lumbar region of the back of the user-wearer, around the sides of the user-wearer, to at least one point adjacent the abdomen of the user-wearer.

Anchor(s) comprise diamond-shaped (triangle-shaped or band-shaped, straight-across, or the like), support system foundations. An anchor provides an ‘anchor’ to attach the elastic retentions to, so to provide the pulling support. The anchor on the front is preferably attached through the support system to the anchor on the back (posterior side). In turn, the posterior side anchors are attached to the backboard technology, providing support from the base of the shirt (garment) upwards. In preferred embodiments the front of the shirt has one anchor which may be diamond-shaped, yet could be shaped any equivalent way so to provide anchorage and support. The back of the shirt has two anchors (a triangle shape and diamond shape, though may have more or less depending upon requirements.) The supporting systems of the upper body need, in turn, to be supported by some sort of measure; the measure is the backboard technology.

The backboard technology comprises a flexible, semi-rigid, or rigid (across the spectrum depending on necessity) material or technology that is built-in, adheres to, is attached to, the lumbar and/or thoracic curvatures of the spine. This material assists in promoting the natural curvature of the low back. With the low back in its natural curvature, the upper body has the proper means to be supported with its systems and/or anchors the backboard—form-fits to lumbar and lower thoracic muscles on the user-wearer. The backboard or spine-align support form-fits to lumbar and lower thoracic vertebrae and musculature on the user-wearer. The lumbar support garment of the posture-support spine-align garment system assists in properly aligning and correcting spine and upper body posture and providing abdominal support during active and in-active periods.

The present invention holds significant improvements and serves as a (posture-support) spine-align garment system. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description. Though the depictions represent an embodiment, the present system may be altered to achieve desired support results.

### BRIEF DESCRIPTION OF THE DRAWINGS

The figures which accompany the written portion of this specification illustrate embodiments and method(s) of use for the present invention, posture-support spine-align garment system, constructed and operative according to the teachings of the present invention.

FIG. 1 shows a perspective view illustrating a posture-support spine-align garment system in an in-use condition showing a relative improvement in posture as realized by a user-wearer according to an embodiment of the present invention.

FIG. 2 is a side perspective view illustrating a lumbar support garment in relation to a spine of the user-wearer according to an embodiment of the present invention of FIG. 1.

FIG. 3 is a side perspective view illustrating an upper body apparel assembly of the lumbar support garment including an elastic retention system and backboard support according to an embodiment of the present invention of FIGS. 1 and 2.

FIG. 4A is a front perspective view illustrating the elastic retention system including flexible stretch bands, and anchor(s) (which both may differ according to body need) according to an embodiment of the present invention of FIG. 1.

FIG. 4B is a front perspective view of a posterior side illustrating the elastic retention system including flexible stretch band(s) and the anchor and showing the backboard, lumbar curve support as it is found on a back of the lumbar support garment in relation to the elastic retention system according to an embodiment of the present invention of FIGS. 1 and 4A.

FIG. 5 is a flowchart illustrating a method of use for the posture-support spine-align garment system according to an embodiment of the present invention of FIGS. 1-4B.

The various embodiments of the present invention will hereinafter be described in conjunction with the appended drawings, wherein like designations denote like elements.

### DETAILED DESCRIPTION

As discussed above, embodiments of the present invention relate to a garment device and more particularly to a posture-support spine-align garment system as used to improve the posture of individual user-wearers. The support systems may differ according to the specific use, garment type, required remedy, performance necessity, gender, and the like.

Generally speaking, the Posture-Support Spine-Align garment (shirt) with “Backboard” Technology is an upper body apparel piece with integrated support systems to assist in properly aligning and stabilizing the lumbar spine and thoracic spine, while providing optimal support of the cervical spine. It may be ideal for individuals who position their spines in “C-posture” (related to upper-cross syndrome—a compromise in the musculoskeletal system which tightens the chest while weakening the back and shoulders, producing a “C” position). The lumbar vertebrae, the largest vertebrae of the spine which naturally resemble a backward “C”, must first be stabilized to provide the upper spine with the proper support. This apparel piece may be designed for desk and manual labor workers, medical and service industry staff, athletes, professional drivers, students, consistent travelers, and individuals with rounded backs and slumping shoulders. It may be specifically fitted for individuals with chronic back and discogenic pain, and can be worn in virtually any arena, including active and inactive environments (performed through changes in the complexities in manufacturing).

The Posture-Support Spine-Align garment (shirt or other) with “Backboard” Technology provides consumers, men, women, and children with a novel restraining garment that offers support to the spinal chamber and upper body. The invention is comprised of a variety of materials depending on the application, including in certain embodiments: compression materials, technology materials, plastics, rubbers, composite material, and heavy, rigid fabrics that provide tensile support. It may be produced in a variety of styles for active apparel such as shirts, vests, jackets for example, inactive apparel, undergarments, therapeutic garments, medical garments, etc. Upon reading this specification, it should be appreciated that, under appropriate circumstances, considering such issues as user preferences, design preference, structural requirements, marketing preferences, cost, available materials, technological advances, etc., other garments and apparel such as, for example, sleeping garments, apparel, shirt, support system, attire, apparel piece, upper body clothing, body suits, concealed garment, non-concealed garment, under garment (for example an undershirt), etc., may be sufficient.

The “Backboard” Technology may comprise a spinal chamber or section made from a synthetic material that will preferably suitably stabilize the spine by neutralizing the vertebrae, pushing them into natural alignment. The “Backboard” may be built into the lumbar and lower thoracic sections of the posture shirt and can conform to the natural curvature of the low and mid spinal regions. The fabric of the lower back side is preferably more rigid with minimal stretching ability as compare to the front side. Due to its shape and limited flexibility (high rigidity) relative to the spine, the “Backboard” can provide the needed resistance to yield a stable lower back immediately and gradually over time. Natural alignment of the spine begins using the present invention at the base with the lumbar. The “Backboard” can be the foundation for the other support system embodiments of the Posture-Support Spine-Align Shirt comprises Posture-Support Spine-Align garment system and is a key component in providing a springboard effect as described below for the upper torso.

The support systems of the Posture-Support Spine-Align Shirt may include V-shaped designs (may vary, may be U-shaped or web-like parallel, cross-patterned, etc.) with rigid fabric on the back side and semi-rigid fabric on the front side, though may differ according to need. The systems may be structured in criss-cross patterns that ‘mimic’ the musculature of the upper body. The diamond support anchors (on front) (triangular on back) may be located in the mid thoracic and lower cervical areas (back), and lower sternum (front), though may be placed in accordance for support. The “Backboard” can be narrow and/or fan outward from the thoracic support anchor on the back side, and may widen towards the lower lumbar section. The anchors can provide bases to which the V-shaped support systems attach (and the elastic, retention support may be U-shaped, X-shaped or the like in certain embodiments.) The shoulder and underarm anchors (optional) can be made from a rigid technology fabric located at the framework piping (optional) along the seams of the garment. The V-shaped support systems can run from the shoulder and underarm anchors to the diamond anchors on both sides of the garment, and can provide pulling and stabilizing of the shoulders, producing the chest-up, head-up position, shoulders-back position (springboard effect). The framework piping may be made from semi-rigid fabric and can follow the seams of the garment, providing the additional foundation to accommodate the support systems.

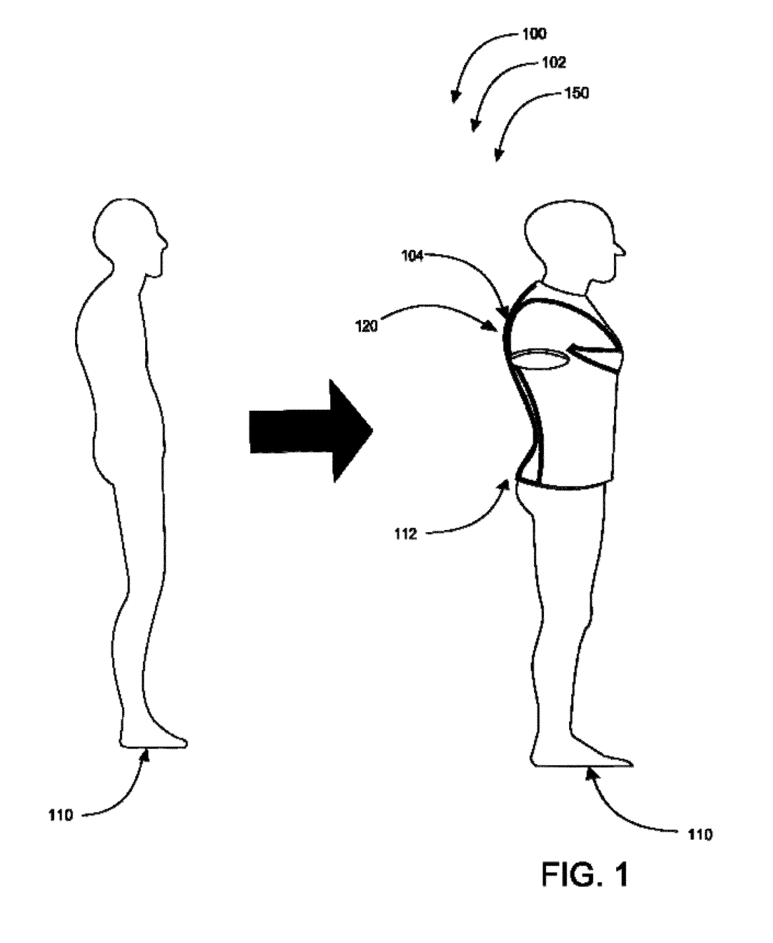

Referring to the drawings by numerals of reference there is shown in FIG. 1, a perspective view illustrating posture-support spine-align garment system 100 in in-use condition 150 showing a relative improvement in posture as realized by user-wearer 110 according to an embodiment of the present invention.

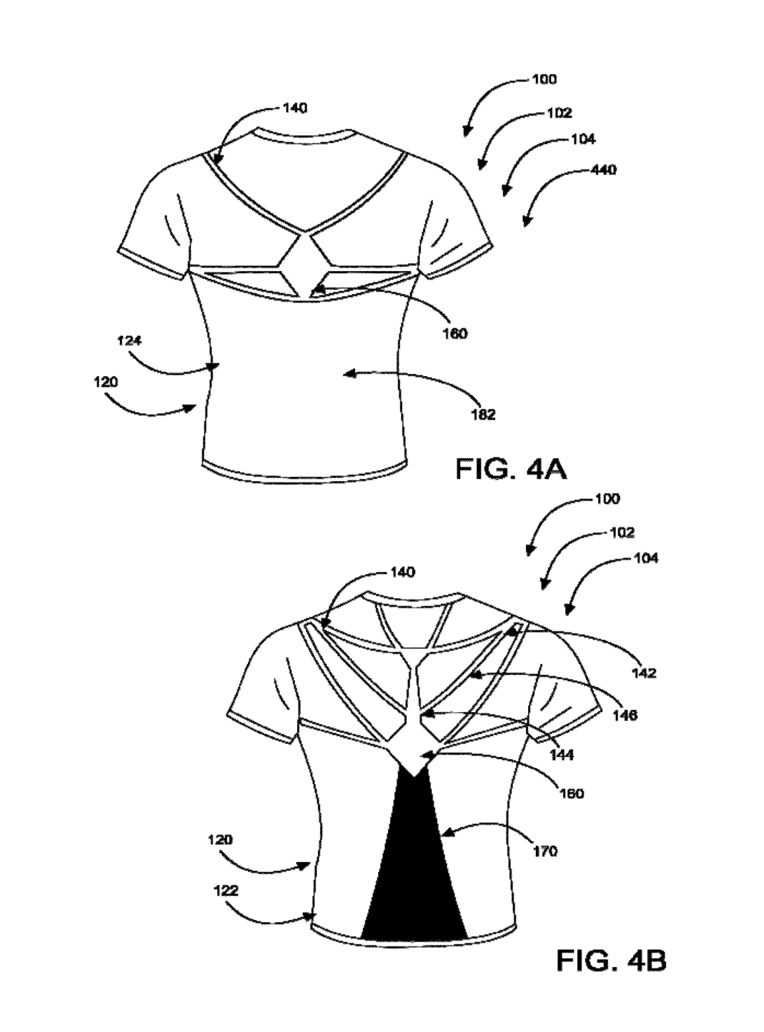

The posture-support spine-align garment system 100 preferably comprises: upper body apparel assembly 120 having elastic retention system 130; flexible stretch bands 140 (having; first end 142; second end 144; and middle portion 146); anchors 160; backboard support 170; front fabric cover 182; and back fabric cover 186. Elastic retention system 130 comprises flexible stretch bands 140 connected to anchors 160, as shown in FIGS. 4A and 4B. Anchors 160 are preferably fixedly anchoring first and second ends (142 and 144, respectively) of flexible stretch bands 140 (for retention) and elastic retention system 130 connected to backboard support 170. First end 142 and second end 144 define limits of middle portion 146 of flexible stretch band(s) 140; wherein middle portion 146 of flexible stretch band(s) 140 terminate with first end 142 and second end 144 (ends that connect to anchors 160.) Elastic retention system 130 is normally in tension and anchors 160 are normally in compression. Elastic retention system 130 controls a relative positioning of a torso of user-wearer 110 as stayed by backboard support 170. Backboard support 170 supports lumbar region 112 of user-wearer 110 and contours to conform to a natural curvature of lumbar region 112. A neck collar may be optionally used with upper body apparel assembly 120 and may be constructed with flexible stretch band(s) 140 in certain embodiments for additional support.

Front fabric cover 182 comprises at least one flexible fabric and back fabric cover 186 comprises a flexible (to rigid) (preferred semi-rigid) fabric in preferred embodiments. In other embodiments flexibility and/or rigidity may vary as per application. Front fabric cover 182 and back fabric cover 186 form a torso cover for upper body apparel assembly 120. Flexible stretch bands 140 of elastic retention system 130 of upper body apparel assembly 120 preferably exerts a tension force on an upper torso of user-wearer 110 and backboard support 170 exerts a compression force on a lower torso of user-wearer 110. In this way (posture-support) spine-align garment system 100 assists in properly aligning and stabilizing lumbar region 112 comprising a lumbar spine and thoracic spine, while providing optimal support of a cervical spine of a spinal cord system.

Posture-support spine-align garment system 100 provides and promotes spine, back, chest, shoulder, neck, head, muscular, posture, anatomical alignment and support in relation to each other and helps to align/straighten the vertebrae of the spinal column and musculature of user-wearer 110. Further, posture-support spine-align garment system 100 decreases nerve and muscle tension in back, via putting body portions needing to be in tension, in tension, and by putting those other body portions needing to be in compression, into compression. Shoulders and neck are assisted in this manner as well to inhibit rounding of shoulders by pulling shoulders backward and downward. The present invention, posture-support spine-align garment system 100 promotes an uplifted position for head, chin, chest, and neck, as shown.

Lumbar support garment 102 comprises upper body apparel assembly 120 and when worn by user-wearer 110 actively resists C-posture (rounding of the back and slumping of the shoulders) in user-wearer 110. This is done by lumbar support garment 102 supporting both a spinal chamber and upper body of user-wearer 110. Upper body apparel assembly 120 is able to be worn during active and inactive periods.

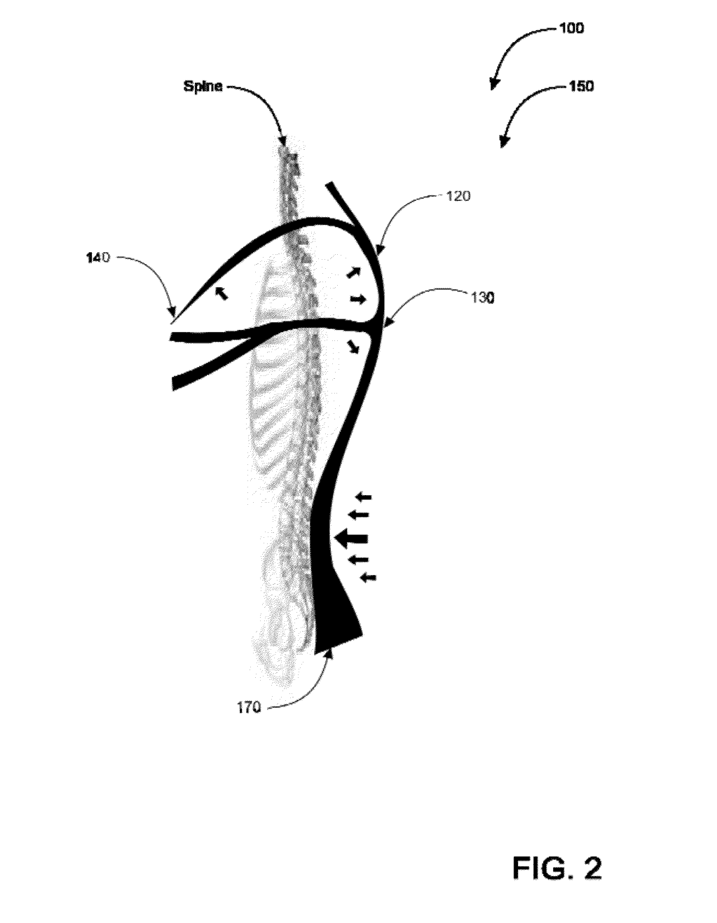

Referring now to FIG. 2, a side perspective view illustrating lumbar support garment 102 in relation to a spine of user-wearer 110 according to an embodiment of the present invention of FIG. 1.

Lumbar support garment 102 of posture-support spine-align garment system 100 preferably comprises a chamber which may be sewn (or otherwise built in, attached to, Velcroed) into a lumbar portion 112 of upper body apparel assembly 120; chamber is centrally-located and conforms to a curvature of the spinal cord system of user-wearer 110. Upper body apparel assembly 120 may further comprise an auxiliary belt that is stretchable and longitudinal sheet-form support bones. The tension and compression is denoted by arrows such as to achieve the desired posture of the spine and body in user-wearer 110.

Lumbar support garment 102 comprises posture shirt 104 in preferred embodiments, and may comprise jackets, body suits, vests or the like as previously mentioned. It should be appreciated that the present invention may be used in virtually any body garment and that the scope of the invention is not to be limited by the garments mentioned herein, but rather that they have been discussed as a means whereby the reader may be suitably enabled to make and use the present invention.

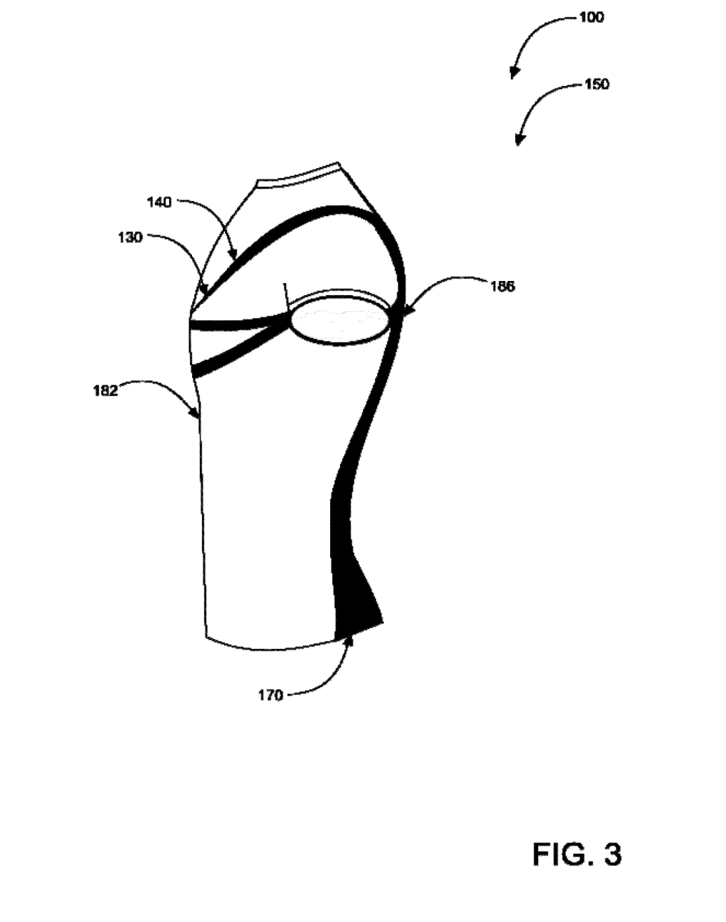

Referring now to FIG. 3, a side perspective view illustrating upper body apparel assembly 120 of lumbar support garment 102 including elastic retention system 130 and backboard support 170 (from front to back of the garment) according to an embodiment of the present invention of FIGS. 1 and 2.

The spinal curve support or Backboard Technology and rear/back of shirt may comprise: tech material, composite, plastic, durable material, heavy fabric, rigid material, flexible material, supportive material. The spinal curve support or Backboard Technology can be sewn into, adhered to, built into, attached to, taped to, part of, in addition to the rear/back of posture shirt 104. Backboard Technology is the spinal piece built into the present invention for support and the thickness, width, height, weight, rigidity may vary with models built for the average man or woman and certain other embodiments or specific uses may be molded for a custom fit specific to user-wearer 110. In certain embodiments the present invention may comprise athletic, medical or casual/non-casual garments.

Upper body apparel assembly 120 may comprises semi-rigid-piping acting as a support-framework and as a mounting-framework; mounting-framework for securing front fabric cover 182 and back fabric cover(s) 186 thereto.

Referring now to FIG. 4A a front perspective view illustrating elastic retention system 130 including flexible stretch band(s) 140 and anchor 160 according to an embodiment of the present invention of FIG. 1.

Upper body apparel assembly 120 comprises posterior side 122 and anterior side 124; wherein posterior side 122 is semi-rigid (or can comprise any level of flexibility/rigidity). Posterior side 122 of upper body apparel assembly 120 comprises brace-like material such as suitable to provide tensile support. Anchor 160 is preferably located adjacent a sternum which may be located lower on female garments to accommodate breasts and higher on male versions to accommodate a substantially flat chest. In this way upper body apparel assembly 120 comprises male and female anatomy concessions so as to better fit and support user-wearers 110. Posterior side 122 of posture shirt 104 may be of more rigid, brace-like material to provide additional support thereby aiding in more intense alignment and less mobility. The retention system and elastic banding may be positioned higher or lower to provide varying methods and degrees for support, and may be anchored (or not) in differing methods to achieve varying results (example: anchored to backboard/spine curve).

Upper body apparel assembly 120 may be worn on the upper body during active and inactive periods which supports the spine to promote proper posture, specifically relating to the curvatures of the lumbar spine and thoracic spine, which, in turn, places the cervical spine in optimal positioning. Upper body apparel assembly 120 may be initialized through semi-rigid piping which may provide the framework of the support and stability for the fabrics of the garment. In conjunction with the piping, a larger semi-rigid (full scope of flexibility/rigidity) section or chamber may be sewn into the lumbar and lower thoracic sections of the apparel piece to provide for correct spinal posture and abdominal support. This chamber is centrally located in the apparel piece, aligned vertically with the spine, and based on the curvature of the spinal cord, specifically the lumbar and lower thoracic. The fabric on the lower posterior side 122 of the piece is rigid, yet stretchable, providing the ultimate support in upper body apparel assembly 120. The fabric on the lower anterior side 124 of the piece may be less rigid allowing this lower section of the garment to expand, so to be pulled over the head and shoulders and worn on the upper body.

FIG. 4B is a front perspective view of the posterior side (backside) of the garment illustrating elastic retention system 130 including flexible stretch band(s) 140 and anchor 160 and showing backboard support 170 as it is found on a back of the lumbar support garment 102 in relation to the elastic retention system 130 according to an embodiment of the present invention of FIGS. 1 and 4A.

Backboard support 170 stabilizes the spinal cord system by neutralizing vertebrae(s) from a cervical to a Coccygeal region, with specific support enhancement to the lumbar and lower thoracic. Front of posture shirt 104 is supportive through a system of supportive anchors 160 bands/attachments, and provides the heads up, chest up/out, shoulders back/down positioning; front (anterior) of shirt support systems attached to rear (posterior) of shirt creating overall system of support. The alignments of the fabrics along the upper portions of the garment preferably coincide with the body's musculature (may be designed with different intentions), assisting in pulling the shoulders backward and downward, reducing shoulder slumping, effectively bringing the upper trunk into proper postural alignment and optimally aligning the cervical spine. The densities of the fabrics that comprise posture shirt 104 may vary and are based on the needs of support. The specific support fabrics may be woven into the piping and spinal chamber of posture shirt 104 assisting in the upper body's control, strength, and stability, aiding with postural and anatomical corrections, providing correct, free movement and optimal performance. Scapula, rhomboids, shoulder blades, latissimus, lumbar spine, thoracic spine, core, trapezius are all supported and directly or indirectly positioned using the present invention. Posture, stability, balance, anatomical, pull, muscle, chest up, head up, control, strength, striation, all may be improved using the present invention. Use of the device is effectively pain free with posture-support spine-align garment system 100 promotes correction of the spinal column (spinal cord system), while still permitting freedom of movement.

The present invention supports the upper body as well as the spine (lumbar, thoracic, and cervical) through use of elastic retention systems 130 which helps support, and provides support. The materials built-in, adhering to, attached to, in conjunction with the front and/or back of the shirt/garment/apparel piece/support system serve to promote and provide spine/back/chest/shoulder/neck/head/muscular/posture/anatomical support. The present invention also provides/promotes a heads-up, chin-up, chest-up/out, shoulders back/down, neck-up healthy posture that benefits user-wearer 110 both medically and promotes comfort by decreasing body stiffness and pain. This is accomplished in part by pinching the shoulders together, posture control, using muscular feedback. This helps to straighten the back, increases mobility and muscular exactness, decreases tension (nerve and muscular) in the back, shoulders, neck, and other body parts. This in turn increases performance, effectively inhibits the rounding of the shoulders, assists with upper-cross syndrome and the like. System of support may also be visible, but yet is aesthetically pleasing such that user-wearers 110 aren't dissuaded from its use in public.

Posture-support spine-align garment system 100 may be sold as kit 440 comprising the following parts: backboard support 170; posture shirt 104; and at least one set of user instructions, or as a whole packaged garment. Posture-support spine-align garment system 100 may be manufactured and provided for sale in a wide variety of sizes and shapes for a wide assortment of applications. Upon reading this specification, it should be appreciated that, under appropriate circumstances, considering such issues as design preference, user preferences, marketing preferences, cost, structural requirements, available materials, technological advances, etc., other kit contents or arrangements such as, for example, male and female versions, including more or less components, customized parts, different color combinations, different support system patterning, parts may be sold separately, etc., may be sufficient.

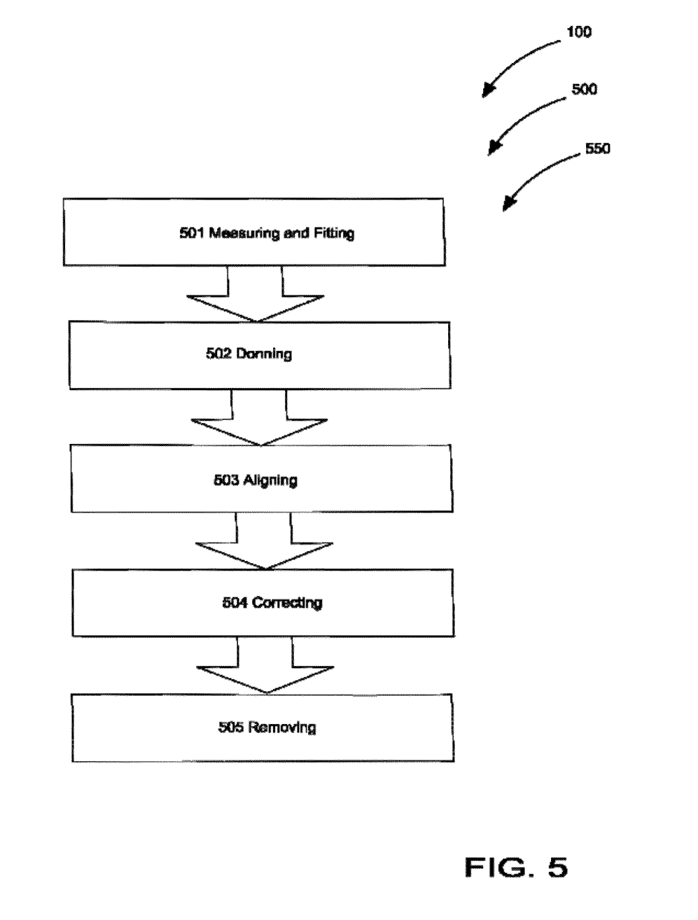

Referring now to FIG. 5, a flowchart 550 illustrating a method of use 500 for posture-support spine-align garment system 100 according to an embodiment of the present invention of FIGS. 1-4B.

Method of use 500 comprises step one 501 measuring and fitting posture shirt 104 to user-wearer 110; step two 502 user-wearer 110 donning posture shirt 104; step three 503 posture shirt 104 aligning body of user-wearer 110; step four 504 correcting body alignment of user-wearer 110. Step five 505 posture shirt 104 may be removed (removing) and changed (changing) out for another posture shirt 104 of higher tension or lesser tension (more or less support), as desired.

It should be noted that step five 505 is an optional step and may not be implemented in all cases. Optional steps of method 500 are illustrated using dotted lines in FIG. 5 so as to distinguish them from the other steps of method 500.

It should be noted that the steps described in the method of use can be carried out in many different orders according to user preference. The use of “step of” should not be interpreted as “step for”, in the claims herein and is not intended to invoke the provisions of 35 U.S.C. §112, ¶6. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A posture-support spine-align garment system comprising:
an upper body apparel assembly having;
an elastic retention system;
flexible stretch bands having;
a first end;
a second end; and
a middle portion;
anchors;
a backboard support;
a front fabric cover;
a back fabric cover;
wherein said elastic retention system comprises said flexible stretch bands connected to said anchors, said anchors fixedly anchoring said first and said second ends of said flexible stretch bands, said elastic retention system connected to said backboard support;
wherein said first end and said second end define limits of said middle portion of said flexible stretch band(s);
wherein said middle portion of said flexible stretch band(s) terminate with said first end and said second end;
wherein said backboard support supports a lumbar region of a user-wearer and contours to conform to a natural curvature of said lumbar region;
wherein said elastic retention system controls a relative positioning of a torso of said user-wearer as stayed by said backboard support;
wherein said front fabric cover comprises a flexible fabric and said back fabric cover comprises a semi-flexible fabric said front fabric;
wherein said front fabric cover and said back fabric covers form a torso cover for said upper body apparel assembly; and
wherein said flexible stretch bands of said elastic retention system of said upper body apparel assembly exerts a tension force on an upper torso of said user-wearer and said backboard support exerts a compression force on a lower torso of said user-wearer; and
wherein said posture-support spine-align garment system assists in properly aligning and stabilizing said lumbar region comprising a lumbar spine and thoracic spine, while providing optimal support of a cervical spine of a spinal cord system.

2. The posture-support spine-align garment system of claim 1 wherein said spine-align garment system provides and promotes spine, back, chest, shoulder, neck, head, muscular, posture, anatomical alignment and support in relation to each other.

3. The posture-support spine-align garment system of claim 2 wherein said spine-align garment system helps to straighten and align said back.

4. The posture-support spine-align garment system of claim 3 wherein said spine-align garment system decreases nerve and muscle tension in said back, said shoulders and said neck.

5. The posture-support spine-align garment system of claim 4 wherein said spine-align garment system is useful to inhibit rounding of said shoulders by pulling said shoulders backward and downward.

6. The posture-support spine-align garment system of claim 2 wherein said spine-align garment system promotes an uplifted position for said head, said chin, said chest, and said neck.

7. The posture-support spine-align garment system of claim 1 wherein said upper body apparel assembly comprises a posterior side and an anterior side, wherein said posterior side is semi-rigid.

8. The posture-support spine-align garment system of claim 7 wherein said posterior side of said upper body apparel assembly comprises brace-like material.

9. The posture-support spine-align garment system of claim 1 wherein said upper body apparel assembly comprise semi-rigid-piping acting as a support-framework and as a mounting-framework, said mounting-framework for securing said front fabric cover and said back fabric covers thereto.

10. The posture-support spine-align garment system of claim 1 further comprising a chamber is sewn into a lumbar portion of said upper body apparel assembly, said chamber centrally-located and conforms to a curvature of said spinal cord system.

11. The posture-support spine-align garment system of claim 1 wherein said upper body apparel assembly is able to be worn during active and inactive periods.

12. The posture-support spine-align garment system of claim 1 wherein said upper body apparel assembly provides tensile support.

13. The posture-support spine-align garment system of claim 10 wherein said backboard support stabilizes the spinal cord system by neutralizing and properly aligning vertebrae(s).

14. The posture-support spine-align garment system of claim 1 wherein said anchor is located adjacent a sternum.

15. The posture-support spine-align garment system of claim 10 wherein curvature of said spinal cord system is directed to a lumbar and thoracic.

16. The posture-support spine-align garment system of claim 15 wherein said upper body apparel assembly comprises male and female anatomy concessions.

17. A posture-support spine-align garment system comprising:
an upper body apparel assembly having;
an elastic retention system;
flexible stretch bands having;
a first end;
a second end; and
a middle portion;
anchors;
a backboard support;
a front fabric cover;
a back fabric cover;
wherein said elastic retention system comprises said flexible stretch bands connected to said anchors, said anchors fixedly anchoring said first and said second ends of said flexible stretch bands, said elastic retention system connected to said backboard support;
wherein said first end and said second end define limits of said middle portion of said flexible stretch band(s);
wherein said middle portion of said flexible stretch band(s) terminate with said first end and said second end;
wherein said backboard support supports a lumbar region of a user-wearer and contours to conform to a natural curvature of said lumbar region;
wherein said elastic retention system controls a relative positioning of a torso of said user-wearer as stayed by said backboard support;
wherein said front fabric cover comprises a flexible fabric and said back fabric cover comprises a semi-flexible fabric said front fabric;
wherein said front fabric cover and said back fabric covers form a torso cover for said upper body apparel assembly;
wherein said spine-align garment system provides and promotes spine, back, chest, shoulder, neck, head, muscular, posture, anatomical alignment and support in relation to each other;
wherein said spine-align garment system helps to straighten and align said back;
wherein said spine-align garment system decreases nerve and muscle tension in said back, said shoulders and said neck;
wherein said spine-align garment system is useful to inhibit rounding of said shoulders by pulling said shoulders backward and downward;
wherein said spine-align garment system promotes an uplifted position for said head, said chin, said chest, and said neck;
wherein said upper body apparel assembly comprises a posterior side and an anterior side, wherein said posterior side is semi-rigid;
wherein said posterior side of said upper body apparel assembly comprises brace-like material;
wherein said upper body apparel assembly is able to be worn during active and inactive periods;
wherein said flexible stretch bands of said elastic retention system of said upper body apparel assembly exerts a tension force on an upper torso of said user-wearer and said backboard support exerts a compression force on a lower torso of said user-wearer; and
wherein said posture-support spine-align garment system assists in properly aligning and stabilizing said lumbar region comprising a lumbar spine and thoracic spine, while providing optimal support of a cervical spine of a spinal cord system.

18. The posture-support spine-align garment system of claim 17 wherein said lumbar support garment when worn by said user-wearer actively resists C-posture in said user-wearer.

19. The posture-support spine-align garment system of claim 17 wherein said lumbar support garment supports both a spinal chamber and upper body of said user-wearer.

20. The posture-support spine-align garment system of claim 17 wherein said lumbar support garment comprises a posture shirt.
